(19) United States
(12) Patent Application Publication
(10) Pub. No.: US 2026/0295144 A1
(43) Pub. Date: Oct. 1, 2026

(54) REDUCTION OF FALSE ALARMS DURING PROVISION OF NEGATIVE PRESSURE WOUND THERAPY

(71) Applicant: T.J.Smith and Nephew,Limited, Hull (GB)

(72) Inventors: Iain Michael Blackburn, York (GB); Yeswanth Gadde, Pocklington (GB); Felix Clarence Quintanar, Hull (GB)

(21) Appl. No.: 19/164,406

(22) PCT Filed: Mar. 21, 2024
(86) PCT No.: PCT/EP2024/057569

§ 371 Date: Sep. 11, 2025

(30) Foreign Application Priority Data
Mar. 27, 2023 (GB) 2304408.4

(51) Int. Cl. A61M 1/00

(52) U.S. Cl. CPC A61M 1/918; A61M 2205/15; A61M 2205/18; A61M 2205/3331; A61M 2205/502

(57) ABSTRACT

A negative pressure wound therapy system can include a negative pressure source configured to provide, via a fluid flow path, negative pressure to a wound, a pressure sensor configured to measure a pressure in the fluid flow path, a user interface configured to provide a plurality of notifications to the user, a first processor configured to operate the negative pressure source based on the pressure and detect an operating condition associated with provision of negative pressure, and a second processor configured to receive from the first processor an indication that the operating condition has been detected during the first time duration. The second processor can be configured to provide, via a user interface, a notification of the operating condition responsive to verifying that a plurality of indications have been received from the first processor during a second time duration that spans a plurality of first time durations.

### TECHNICAL FIELD

Embodiments described herein relate to apparatuses, systems, and methods for the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

A negative pressure wound therapy system can include a negative pressure source configured to provide, via a fluid flow path, negative pressure to a wound covered by a wound dressing. The system can include a pressure sensor configured to measure a pressure in the fluid flow path. The system can include a user interface configured to receive input from a user and provide a plurality of notifications to the user. The system can include a first processor configured to operate the negative pressure source based on the pressure in the fluid flow path and detect an operating condition associated with provision of negative pressure. The system can include a second processor configured to, upon expiration of a first time duration, receive from the first processor an indication that the operating condition has been detected during the first time duration. The second processor can be configured to, via a user interface, provide a notification of the operating condition to the user in response to verifying that a plurality of indications have been received from the first processor during a second time duration that spans a plurality of first time durations.

The negative pressure wound therapy system of any of the preceding paragraphs and/or any of the systems, apparatuses, or devices disclosed herein can include one or more of the following features. Providing the notification of the operating condition to the user in response to verifying that the plurality of indications have been received from the first processor during the second time duration can reduce incidence of false alarms. The system can include a canister configured to be positioned in the fluid flow path and further configured to store fluid aspirated from the wound. The operating condition can include detection that the canister is full. The first processor can be configured to detect that the canister is full in response to the pressure over the first time duration satisfying a pressure threshold. The second processor can be configured to continue provision of the notification of the operating condition in response to receiving from the first processor the indication subsequent to expiration of the second time duration. The second processor can be configured to stop provision of the notification of the operating condition in response to no longer receiving from the first processor the indication subsequent to expiration of the second time duration. The first time duration can include 1 minute, and the second time duration can include 30 minutes. The indication that the operating condition has been detected during the first time duration can be received by the second processor via a serial interface.

A negative pressure wound therapy system can include a negative pressure source configured to provide, via a fluid flow path, negative pressure to a wound covered by a wound dressing. The system can include a pressure sensor configured to measure a pressure in the fluid flow path. The system can include a user interface configured to receive input from a user and provide a plurality of notifications to the user. The system can include a first processor configured to operate the negative pressure source based on the pressure in the fluid flow path and detect an operating condition associated with provision of negative pressure. The system can include a second processor configured to operate the user interface and communicate with the first processor. The second processor can be configured to, based on the pressure in the fluid flow path, verify detection of the operating condition by the first processor. The second processor can be configured to provide, via the user interface, a notification of the operating condition to the user in response to verifying the operating condition. The second processor can be configured to override detection of the operating condition by the first processor in response to not verifying the operating condition.

The negative pressure wound therapy system of any of the preceding paragraphs and/or any of the systems, apparatuses, or devices disclosed herein can include one or more of the following features. Providing the notification of the operating condition to the user in response to verifying that operating condition can improve sensitivity of the notification of the operating condition. Overriding detection of the operating condition includes not providing the notification of the operating condition to the user. The operating condition can include at least one of a leak in the fluid flow path detected in response to the pressure in the fluid flow path not satisfying a pressure threshold over a duration of time; or a blockage in the fluid flow path detected in response to flow in the fluid flow path no longer satisfying a flow threshold; or a canister full condition indicative of a level of fluid in a canister satisfying a fluid level threshold. The first processor can be configured to detect the canister full condition in response to a determination that the pressure in the fluid flow path over a duration of time satisfies a pressure threshold. The system can include a housing supporting the negative pressure source and the first processor. The second processor can be supported by the housing or be remote to the housing. The operating condition can be communicated to the second processor via a serial interface. The operating condition can be detected periodically within a specified duration of time.

The negative pressure wound therapy system of any of the preceding paragraphs and/or any of the systems, apparatuses, or devices disclosed herein can include one or more of the following features. The second processor can be configured to verify the operating condition based on a detection of one or more environmental changes. The one or more environmental changes can include a change in altitude. In response to not verifying the operating condition, the second processor can be configured to provide an indication to the first processor and cause the first processor to clear detection of the operating condition. In response to not verifying detection of the operating condition, the second processor can be further configured to reset the first processor.

Disclosed herein are methods of operating a negative pressure wound therapy systems of any of the preceding paragraphs and/or any of the systems, devices, or apparatuses disclosed herein.

Disclosed herein are kits that include the negative pressure wound therapy system of any of the preceding paragraphs and/or any of the systems, devices, or apparatuses disclosed herein and one or more wound dressings.

Disclosed herein are kits that include the negative pressure wound therapy system of any of the preceding paragraphs and/or any of the systems, devices, or apparatuses disclosed herein and one or more canisters.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1 A illustrates a negative pressure wound therapy system.

FIG. 1B illustrates another negative pressure wound therapy system.

FIG. 2A is an isometric view of a negative pressure wound therapy device and canister, showing the canister detached from the pump assembly of the device.

FIG. 2B is a back view of the negative pressure wound therapy device shown in FIG. 2A.

FIG. 2C illustrates a top surface of the negative pressure wound therapy device shown in FIG. 2A, showing a user interface.

FIG. 3 illustrates a schematic of a control system of a negative pressure wound therapy device.

FIG. 4 illustrates another negative pressure wound therapy system.

FIG. 5A illustrates another negative pressure wound therapy device and canister.

FIG. 5B illustrates a back view of the negative pressure wound therapy device and canister of FIG. 5A.

FIG. 6 illustrates a graph of pressure pulses over time.

FIG. 7 illustrates a flow diagram of a verification and filtering process of an operating condition.

### DETAILED DESCRIPTION

Embodiments disclosed herein relate to systems and methods of treating and/or monitoring a wound. Some embodiments of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, bums, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760−X) mmHg. In addition, negative pressure that is “less” or “smaller” than X mmHg corresponds to pressure that is closer to atmospheric pressure (for example, −40 mmHg is less than −60 mmHg). Negative pressure that is “more” or “greater” than −X mmHg corresponds to pressure that is further from atmospheric pressure (for example, −80 mmHg is more than −60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, or methods of efficiently adjusting TNP prescriptions and delivering effective TNP therapy.

### Wound Therapy System

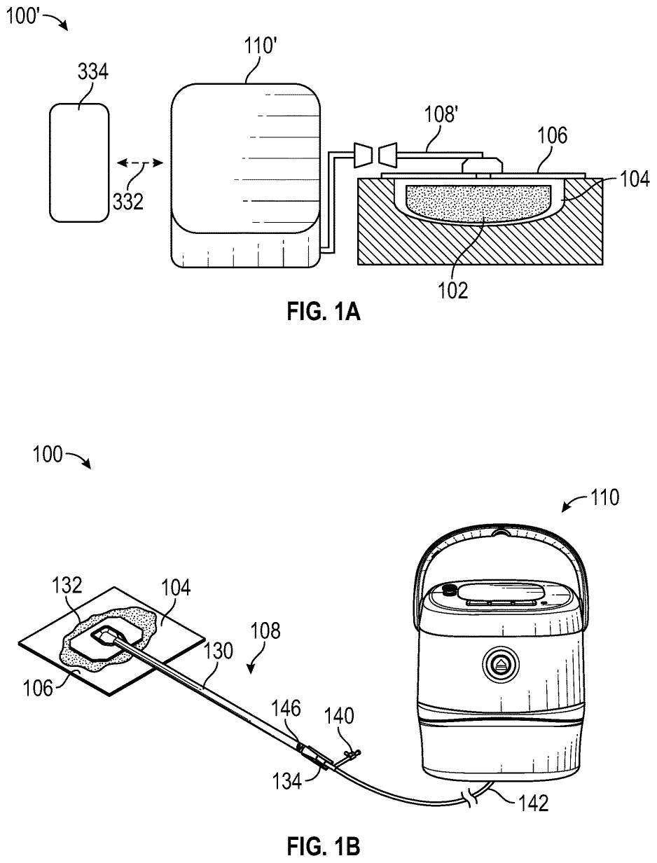

FIG. 1A schematically illustrates a negative pressure wound treatment system 100′ (sometimes referred to as a reduced or negative pressure wound therapy system, a TNP system, or a wound treatment system). In any implementations disclosed herein, though not required, the negative pressure wound treatment system 100′ can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108′ (also referred to herein as a flexible suction adapter or a fluidic connector) can be used to connect the wound cover 106 with a wound therapy device 110′ (sometimes as a whole or partially referred to as a “pump assembly”) configured to supply reduced or negative pressure. The conduit 108′ can be a single or multi lumen tube. A connector can be used to removably and selectively couple a conduit or tube of the wound therapy device 110′ with the conduit 108′.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108′ or any other conduit disclosed herein can be formed from polyurethane, PVC, nylon, polyethylene, silicone, or any other suitable material.

The wound cover 106 can have a port (not shown) configured to receive an end of the conduit 108′. In some cases, the conduit 108′ can otherwise pass through or under the wound cover 106 to supply reduced pressure to the wound 104 so as to maintain a desired level of reduced pressure in the wound 104. The conduit 108′ can be any suitable article configured to provide at least a substantially sealed fluid flow pathway or path between the wound therapy device 110′ and the wound cover 106, so as to supply the reduced pressure provided by the wound therapy device 110′ to wound 104.

The wound cover 106 and the wound filler 102 can be provided as a single article or an integrated single unit. In some cases, no wound filler is provided and the wound cover by itself may be considered the wound dressing. The wound dressing can then be connected, via the conduit 108′, to a source of negative pressure of the wound therapy device 110′. In some cases, though not required, the wound therapy device 110′ can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110′ can operate with or without the use of an exudate canister. In some cases, as is illustrated, the wound therapy device 110′ can include an exudate canister. In some cases, configuring the wound therapy device 110′ and conduit 108′ so that the conduit 108′ can be quickly and easily removed from the wound therapy device 110′ can facilitate or improve the process of wound dressing or pump changes, if necessary. Any of the pump assemblies disclosed herein can have any suitable connection between the conduit 108′ and the pump.

The wound therapy device 110′ can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 110′.

As will be described in greater detail below, the negative pressure wound treatment system 100′ can be configured to provide a connection 332 to a separate or remote computing device 334. The connection 332 can be wired or wireless (such as, Bluetooth, Bluetooth low energy (BLE), Near-Field Communication (NFC), WiFi, or cellular). The remote computing device 334 can be a smartphone, a tablet, a laptop or another standalone computer, a server (such as, a cloud server), another pump device, or the like.

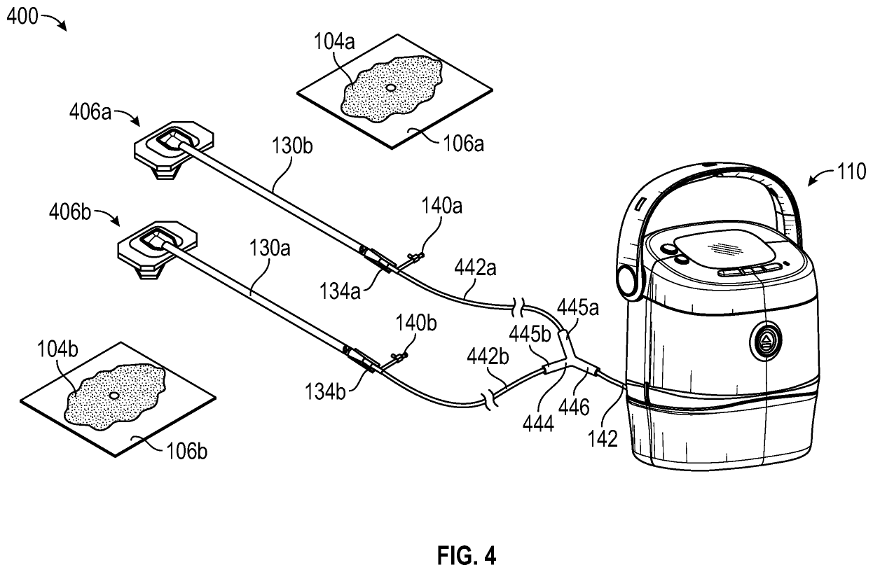

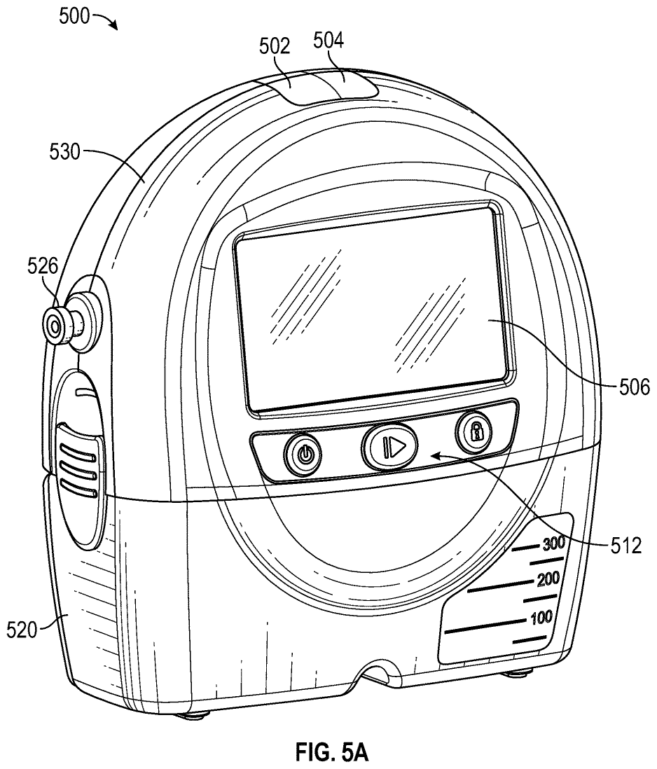

FIG. 1B illustrates another negative pressure wound treatment system 100. The negative pressure wound treatment system 100 can have any of the components, features, or other details of any of the other negative pressure wound treatment system disclosed herein, including without limitation the negative pressure wound treatment system 100 illustrated in FIG. 1A or the negative pressure wound treatment system 400 illustrated in FIG. 4, in combination with or in place of any of the components, features, or other details of the negative pressure wound treatment system 100 shown in FIG. 1B and/or described herein. The negative pressure wound treatment system 100 can have a wound cover 106 over a wound 104 that can seal the wound 104. A conduit 108, such as a single or multi lumen tube can be used to connect the wound cover 106 with a wound therapy device 110 (sometimes as a whole or partially referred to as a “pump assembly”) configured to supply reduced or negative pressure. The wound cover 106 can be in fluidic communication with the wound 104.

With reference to FIG. 1B, the conduit 108 can have a bridge portion 130 that can have a proximal end portion and a distal end portion (the distal end portion being closer to the wound 104 than the proximal end portion, and an applicator 132 at the distal end of the bridge portion 130 forming the flexible suction adapter (or conduit) 108. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108 can be a Soft Port manufactured by Smith & Nephew. As mentioned, the negative pressure wound treatment system 100 can include a source of negative pressure, such as the wound therapy device 110, capable of supplying negative pressure to the wound 104 through the conduit 108. Though not required, the wound therapy device 110 can also include a canister or other container for the storage of wound exudates and other fluids that can be removed from the wound.

The wound therapy device 110 can be connected to the connector 134 via a conduit or tube 142. In use, the applicator 132 can be placed over an aperture formed in a wound cover 106 that is placed over a suitably-prepared wound or wound 104. Subsequently, with the wound therapy device 110 connected via the tube 142 to the connector 134, the wound therapy device 110 can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge portion 130 and can each preferably comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970.RTM., or Gehring 879.RTM.) or a nonwoven material, or terry-woven or loop-pile materials. The fibers may not necessarily be woven, and can include felted and flocked (including materials such as Flotex. RTM.) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge portion 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108 can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. Preferably, the air leak 146 is located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 μm. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic. In some cases, the gas leak 146 can supply a relatively constant gas flow that does not appreciably increase as additional negative pressure is applied to the conduit 108. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values.

In operation, the wound filler 102 can be inserted into the cavity of the wound 104, and wound cover 106 can be placed so as to seal the wound 104. The wound therapy device 110 can provide negative pressure to the wound cover 106, which can be transmitted to the wound 104 via the wound filler 102. Fluid (such as, wound exudate) can be drawn through the conduit 108 and stored in a canister. In some cases, fluid is absorbed by the wound filler 102 or one or more absorbent layers (not shown).

Wound dressings that can be utilized with the pump assembly and systems of the present application include Renasys-F™, Renasys-G™, Renasys AB™, and Pico™ Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325, U.S. Pat. No. 9,084,845, and International Patent Publication No. WO2021/069642, each of which is incorporated by reference in its entirety as if fully set forth herein. In some cases, other suitable wound dressings can be utilized.

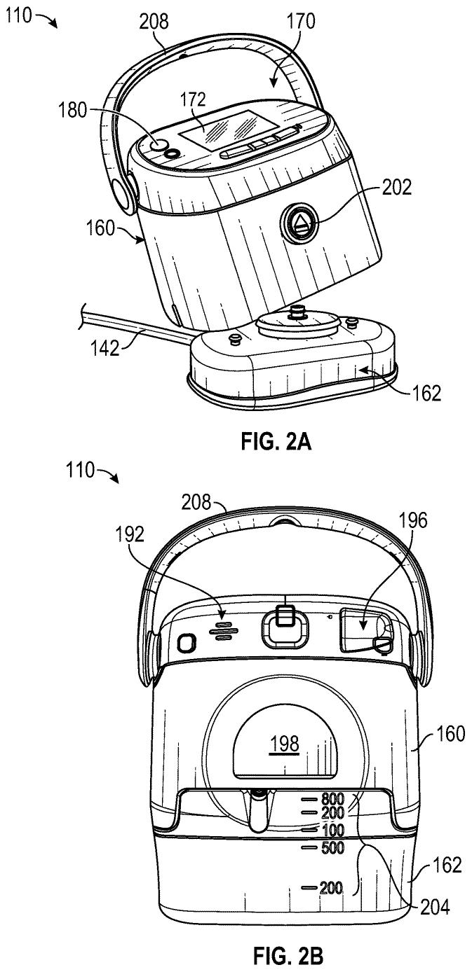

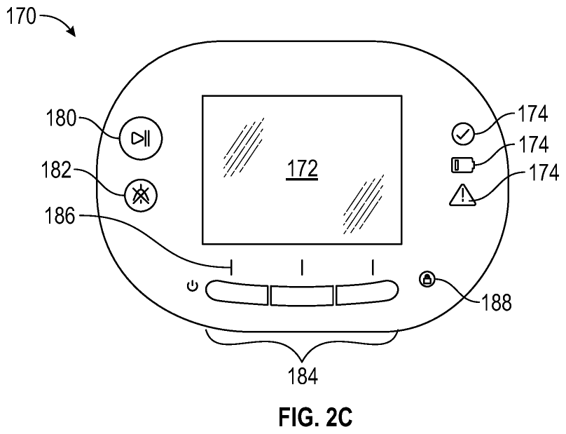

FIGS. 2A-2C show the negative pressure wound therapy device 110. As illustrated, a pump assembly 160 and canister 162 can be connected, thereby forming the wound therapy device 110. With reference to FIG. 2C, the pump assembly 160 can include an interface panel 170 having a display 172, one or more indicators 174, or one or more controls or buttons, including, for example and without limitation, a therapy start and pause button 180 or an alarm/alert mute button 182. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

Additionally, the interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. For example, therapy setting adjustment can be locked/unlocked via the lock/unlock control 188. When the lock/unlock button 188 is in the locked state, depressing one or more of the various other buttons or the display will not cause the pump assembly 160 to change any display functions or performance functions of the device. This way, the interface panel 170 will be protected from inadvertent bumping or touching of the various buttons or display. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and or a status of the pump. For example and without limitation, the one or more indicators 174 can be configured to provide a visual indication of a status of the pump assembly 160 or other components of the negative pressure wound treatment system 100, including without limitation the conduit 108 or the wound cover 106 (such as, to provide an indication of normal operation, low battery, a leak, canister full, blockage, overpressure, or the like). Any one or more suitable indicators can be additionally or alternatively used, such as visual, audio, tactile indicator, and so on.

FIG. 2B shows a back or rear view of the wound therapy device 110 shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack 196 for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. For example, the canister 162 can have an 800 mL (or approximately 800 mL) capacity, or from a 300 mL or less capacity to a 1000 mL or more capacity, or any capacity level in this range. The canister 162 can include a tubing for connecting to the conduit 108 in order to form a fluid flow path. The canister 162 can be replaced with another canister, such as when the canister 162 has been filled with fluid. With reference to FIG. 2A, the wound therapy device 110 can include a canister inlet tube 142 (also referred to herein as a dressing port connector) in fluid communication with the canister 162. For example and without limitation, the canister inlet tube 142 can be used to connect with the conduit 108.

The canister 162 can be selectively coupleable and removable from the pump assembly 160. With reference to FIG. 2A, in some cases, a canister release button 202 can be configured to selectively release the canister 162 from the pump assembly 160. With reference to FIG. 2B, the canister 162 can have one or more fill lines or graduations 204 to indicate to the user and amount of fluid or exudate stored within the canister 162.

The wound therapy device 110 can have a handle 208 that can be used to lift or carry the wound therapy device 110. The handle 208 can be coupled with the pump assembly 160 and can be rotatable relative to the wound therapy device 110 so that the handle can be rotated upward for lifting or carrying the wound therapy device 110 or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110.

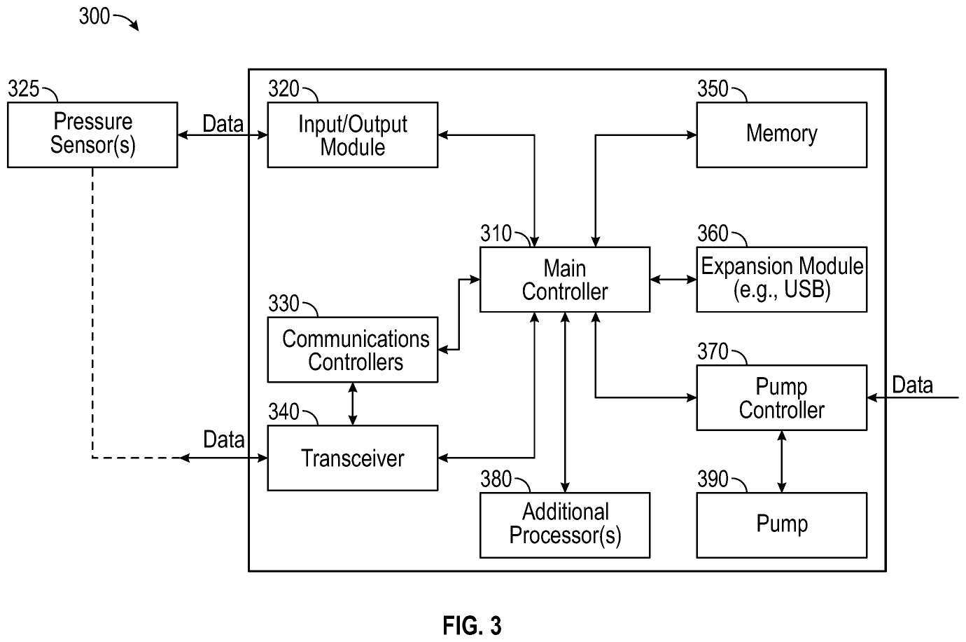

FIG. 3 illustrates a schematic of a control system 300 that can be employed in any of the wound therapy devices described herein, such as in the wound therapy device 110. Electrical components can operate to accept user input, provide output to the user, operate the pressure source, provide connectivity, and so on. A first processor (such as, a main controller 310) can be responsible for user activity, and a second processor (such as, a pump controller 370) can be responsible for controlling another device, such as a pump 390.

An input/output (I/O) module 320 can be used to control an input and/or output to another component or device, such as the pump 390, one or more sensors (for example, one or more pressure sensors 325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 340 over a wireless connection.

The main controller 310 can receive data from and provide data to one or more expansion modules 360, such as one or more USB ports, SD ports, Compact Disc (CD) drives, DVD drives, FireWire ports, Thunderbolt ports, PCI Express ports, and the like. The main controller 310, along with other controllers or processors, can store data in memory 350 (such as one or more memory modules), which can be internal or external to the main controller 310. Any suitable type of memory can be used, including volatile or non-volatile memory, such as RAM, ROM, magnetic memory, solid-state memory, Magnetoresistive random-access memory (MRAM), and the like.

The main controller 310 can be a general purpose controller, such as a low-power processor or an application specific processor. The main controller 310 can be configured as a “central” processor in the electronic architecture of the control system 300, and the main controller 310 can coordinate the activity of other processors, such as the pump controller 370, one or more communications controllers 330, and one or more additional processors 380. The main controller 310 can run a suitable operating system, such as a Linux, Windows CE, VxWorks, etc.

The pump controller 370 can control the operation of a pump 390, which can generate negative or reduced pressure. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 370 can measure pressure in a fluid flow path, using data received from one or more pressure sensors 325, calculate the rate of fluid flow, and control the pump. The pump controller 370 can control the pump actuator (such as, a motor) so that a desired level of negative pressure is achieved in the wound 104. The desired level of negative pressure can be pressure set or selected by the user. The pump controller 370 can control the pump (for example, pump motor) using pulse-width modulation (PWM) or pulsed control. A control signal for driving the pump can be a 0-100% duty cycle PWM signal. The pump controller 370 can perform flow rate calculations and detect alarms. The pump controller 370 can communicate information to the main controller 310. The pump controller 370 can be a low-power processor.

Any of the one or more communications controllers 330 can provide connectivity (such as, a wired or wireless connection 332). The one or more communications controllers 330 can utilize one or more transceivers 340 for sending and receiving data. The one or more transceivers 340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 330 can provide one or more of the following types of connections: Global Positioning System (GPS), cellular connectivity (for example, 2G, 3G, LTE, 4G, 5G, or the like), NFC, Bluetooth connectivity (or BLE), radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 330 can provide dual GPS/cellular functionality. Cellular functionality can, for example, be 3G, 4G, or 5G functionality. The one or more communications controllers 330 can communicate information to the main controller 310. Any of the one or more communications controllers 330 can include internal memory or can utilize memory 350. Any of the one or more communications controllers 330 can be a low-power processor.

The control system 300 can store data, such as GPS data, therapy data, device data, and event data. This data can be stored, for example, in memory 350. This data can include patient data collected by one or more sensors. The control system 300 can track and log therapy and other operational data. Such data can be stored, for example, in the memory 350.

Using the connectivity provided by the one or more communications controllers 330, the control system 300 can upload any of the data stored, maintained, or tracked by the control system 300 to a remote computing device, such as the device 334. The control system 300 can also download various operational data, such as therapy selection and parameters, firmware and software patches and upgrades, and the like (for example, via the connection to the device 334). The one or more additional processors 380, such as processor for controlling one or more user interfaces (such as, one or more displays), can be utilized. In some cases, any of the illustrated or described components of the control system 300 can be omitted depending on an embodiment of a wound monitoring or treatment system in which the control system 300 is used.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Multiple Dressing Negative Wound Therapy

FIG. 4 illustrates another negative pressure wound treatment system 400. The system 400 can include a wound therapy device capable of supplying negative pressure to the wound site or sites, such as wound therapy device 110. The wound therapy device 110 can be in fluidic communication with one or more wound dressings 406a, 406b (collectively referred to as 406) so as to supply negative pressure to one or more wounds, such as the wounds 104a and 104b. A first fluid flow path can include components providing fluidic connection from the wound therapy device 110 to the first wound dressing 406a. As a non-limiting example, the first fluid flow path can include the path from the wound dressing 406a to the wound therapy device 110 or the path from the first wound dressing 406a to an inlet 446 of a branching attachment (or connector) 444 in fluidic connection with the wound therapy device 110. Similarly, a second fluid flow path can include components providing fluidic connection from the wound therapy device 110 to the second wound dressing 406b.

The system 400 can be similar to the system 100 with the exception that multiple wounds 104a and 140b are being treated by the system 400. The system 400 can include any one or more of the components of the system 100, which are illustrated in FIG. 4 with appended letter “a” or “b” to distinguish between the first and second wounds (such as, the wounds 104a and 104b, the covers 106a and 106b). As illustrated, the system 400 can include a plurality of wound dressings 406a, 406b (and corresponding fluid flow paths) in fluidic communication with the wound therapy device 110 via a plurality of suction adapters, such as the adapter 108. The suction adapters can include any one or more of the components of the adapter 108, which are illustrated in FIG. 4 with appended letter “a” or “b” to distinguish between the first and second wounds (such as, the bridge portions 130a and 130b, the connectors 134a and 134b, and the caps 140a and 140b).

The wound therapy device 110 can be fluidically coupled via the tube 142 with the inlet 446 of the connector 444. The connector 444 can be fluidically coupled via branches 445a, 445b and tubes or conduits 442a, 442b with the connectors 134a, 134b, which can be fluidically coupled with the tubes or conduits 130a, 130b. The tubes or conduits 130a, 130b can be fluidically coupled with the wound dressings 406a, 406b. Once all conduits and dressing components are coupled and operably positioned, the wound therapy device 110 can be activated, thereby supplying negative pressure via the fluid flow paths to the wounds 104a, 104b. Application of negative pressure can be applied until a desired level of healing of the wounds 104a, 104b is achieved. Although two wounds and wound dressing are illustrated in FIG. 4, some implementations of the wound therapy device 110 can provide treatment to a single wound (for instance, by closing the unused branch 445a or 445b of the connector 444) or to more than two wounds (for instance, by adding branches to the connector 444).

The system 400 can include one or more features disclosed in U.S. Patent Publication No. 2020/0069850, International Publication No. WO2018/167199, International Publication No. WO2018/167199, or International Publication No. WO2023/072704, each of which is incorporated by reference in its entirety.

### Another Wound Therapy System

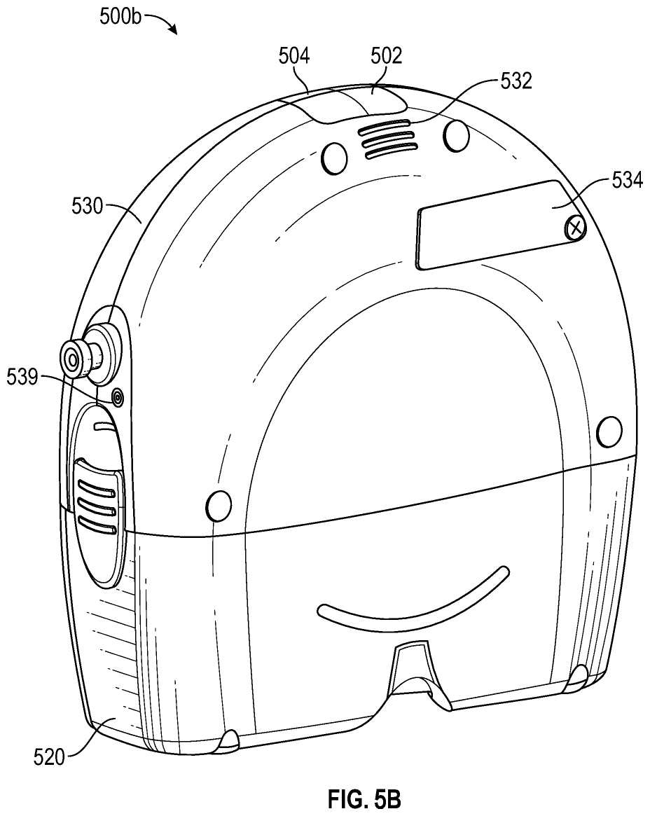

FIGS. 5A and 5B illustrates another negative pressure wound treatment system 500 (or negative pressure wound therapy device), which can be similar to the system 100. The system 500 can include a pump assembly 530 and a canister 520. As illustrated, the pump assembly 530 and the canister 520 can be connected, thereby forming the wound therapy system 500. The pump assembly 530 can include one or more indicators, such as visual indicator 502 configured to indicate alarms and visual indicator 504 configured to indicate status of the pump assembly 530. The visual indicators 502 and 504 can alert (or alarm) a user (for example, patient, health care provider, or the like) to a variety of operating or failure conditions of the pump assembly 530, including alerting the user to normal or proper operating conditions, pump failure, power supplied to the pump or power failure, detection of a leak within the wound cover or flow pathway (sometimes referred to as fluid flow path), suction blockage in the flow pathway, canister full, overpressure, or any other similar or suitable conditions or combinations thereof. Any one or more suitable indicators can be additionally or alternatively used, such as visual, audio, tactile indicator, and so on.

The pump assembly 530 can include a display 506 (such as a screen) mounted in a recess formed in a case of the pump assembly 530. The display 506 can be a touch screen display. The display 506 can support playback of audiovisual (AV) content, such as instructional videos, and render a number of screens or graphical user interfaces (GUIs) for configuring, controlling, and monitoring the operation of the pump assembly 530. The pump assembly 530 can include one or more strap mounts 526 for connecting a carry strap to the pump assembly 530 or for attaching a cradle. The canister 520 may be replaced with another canister, such as when the canister 520 has been filled with fluid.

The pump assembly 530 can include buttons 512 (such as keys) that allow the user to operate and monitor the operation of the pump assembly 530. One of the buttons 512 can operate as a power button to turn on/off the pump assembly 530. Another of the buttons 512 can operate as a play/pause button for the delivery of therapy.

The canister 520 can hold fluid (such as, exudate) removed from the wound cavity 104. The canister 520 includes one or more latches for attaching the canister to the pump assembly 530. For example, the canister 520 as illustrated can have a capacity of 300 mL and include graduations. The canister 520 can include a tubing channel for connecting to the conduit 142.

FIG. 5B illustrates a rear view 500B of the pump assembly 530 and the canister 520. The pump assembly 530 can include a speaker 532 for producing sound. The speaker 532 can generate an acoustic alarm in response to deviations in therapy delivery, non-compliance with therapy delivery, or any other similar or suitable conditions or combinations thereof.

The pump assembly 530 can include a filter access door 534 for accessing and replacing one or more filters, such as antibacterial filters. The pump assembly 530 can comprise a power jack 539 for charging and recharging an internal battery of the pump assembly. The pump assembly 530 can include a disposable power source, such as batteries, so that no power jack is needed. The system 500 can include 300 a control system, such as the control system 300.

### Reduction of False Alarms

As disclosed herein, any of the negative pressure wound therapy systems can monitor and alert (or alarm) the user to a variety of operating or failure conditions, such as leak in the fluid flow path, blockage in the fluid flow path, canister full, dressing full, overpressure, or the like. As disclosed herein, the one or more operating conditions can be detected by the pump controller 370. In some cases, the pump controller 370 can communicate detection of the one or more operating conditions to the main controller 310, which can provide one or more notifications (or alarms) to the user. This may lead to undesired provision of one or more false alarms particularly when the pump controller 370 has limited processing power, memory, or other hardware resources and, as a result, is unable to implement sophisticated processes (which can be implemented in software or firmware) for verifying the correctness of detection of the one or more operating conditions. In some implementations, the main controller 310 can verify or otherwise “filter” the detection of the one or more operating conditions by the pump controller 370 prior to providing the one or more notifications to the user. Filtering can refer to filtering out one or more erroneous detections of the one or more operating conditions (for instance, due to one or more errant pressure measurements). This approach can be particularly advantageous when the pump controller 370 has limited processing power, memory, or other hardware resources, but the main controller 310 does not suffer from such deficiencies. As a result, the main controller 310 can implement more sophisticated processes for verifying or filtering the correctness of detection of the one or more operating conditions. Advantageously, this can lead to reduction of false alarms (or improvement of the sensitivity of alarms and improvement of the provision of negative pressure wound therapy.

In some cases, the verification or filtering can be additionally or alternatively performed remotely from the negative pressure wound therapy system. For instance, one or more remote computing devices can perform the verification or filtering. Such one or more remote computing devices can be located in the cloud. Any references to the main controller 310 can be contemplated as substituting the main controller 310 for one or more remote controllers or including one or more remote controllers in the processing.

The pump controller 370 can be responsible for controlling the negative pressure source to deliver negative pressure wound therapy in accordance with a therapy prescription. The pump controller 370 can detect the one or more operating conditions and provide indication of such detection to the main controller 310. The main controller 310 can operate the user interface, including receiving user commands, providing (where applicable) user commands to the pump controller 370, and providing one or more notifications of the one or more operating conditions to the user. To improve safety and reduce incidence of false alarms, the main controller 310 can implement verification or filtering of the one or more operating conditions detected by the pump controller 370 before providing the one or more notifications to the user. Such verification or filtering can be performed in software or firmware.

The verification or filtering can include counting the number of notifications of an operating condition received from the pump controller 370 and providing a notification to the user in response to the count satisfying threshold. Such threshold can be a number (such as, 5 consecutive notifications) that is indicative of a time duration over which the presence of the operating condition has been detected.

The verification or filtering can include the main controller 310 independently detecting an operating condition and either confirming the detection by the pump controller 370 or overriding such detection. For instance, the main controller 310 can receive (directly or from the pump controller 370) one or more of pressure in the fluid flow path measured by one or more pressure sensors or level of activity of the negative pressure source (such as, pump speed measured by a tachometer or a duty cycle measurement) and use such one or more measurements to independently detect presence of the one or more operating conditions. The main controller 310 can verify that the pump controller 370 correctly detected the operating condition and provide a notification to the user or override an erroneous detection by the pump controller 370. The verification or filtering can be performed in parallel or redundantly with the detection of the one or more operating conditions by the pump controller 370. Such independent verification or filtering can be performed at all times or periodically (for instance, as a spot check to ensure that the pump controller 370 is operating correctly). Advantageously, such parallel processing by the main controller 310 and the pump controller 370 can speed up provision of any alarms.

The main controller 310 can communicate with the pump controller 370 using a serial communication protocol. For instance, I2C, SPI, or USB protocols among others can be used. The main controller 310 can communicate with the pump controller 370 using a parallel communication protocol.

For an operating condition that may compromise patient's safety (such as, overpressure) and necessitates pausing the delivery of therapy, verification or filtering may be skipped or performed quickly (for instance, by setting a small threshold number, such as 2).

Provision of negative pressure wound therapy can be affected by one or more environmental changes, such as, atmospheric pressure, temperature, or vibration. For example, the one or more environmental changes can be due to travel in an aircraft (such as, an airplane or helicopter). The main controller 310 can monitor such one or more environmental changes and perform the verification or filtering based on the one or more environmental changes. Monitoring the one or more environmental changes can be performed using one or more sensors, such as altimeters, inertial measurement units (IMUs), accelerometers, gyroscopes, or the like. As an example of verification or filtering based on the one or more environmental changes, travel in an aircraft can introduce radio frequency (RF) noise into, for instance, pressure monitored by one or more pressure sensors. As a result, indication of the one or more operating conditions to the user may be suspended or delayed (for instance, by increasing the threshold number) in response to detecting that the patient is travelling in the aircraft. As another example, travel in an aircraft can cause rapid changes in atmospheric pressure, which may cause an overpressure condition in the fluid flow path and may compromise patient's safety. In order to promote patient safety while achieving the goal of continuing to provide negative pressure wound therapy, the main controller 310 can lower the set point to a safe value (such as, −25 mmHg) while it is detected that in response to detecting that the patient is travelling in the aircraft.

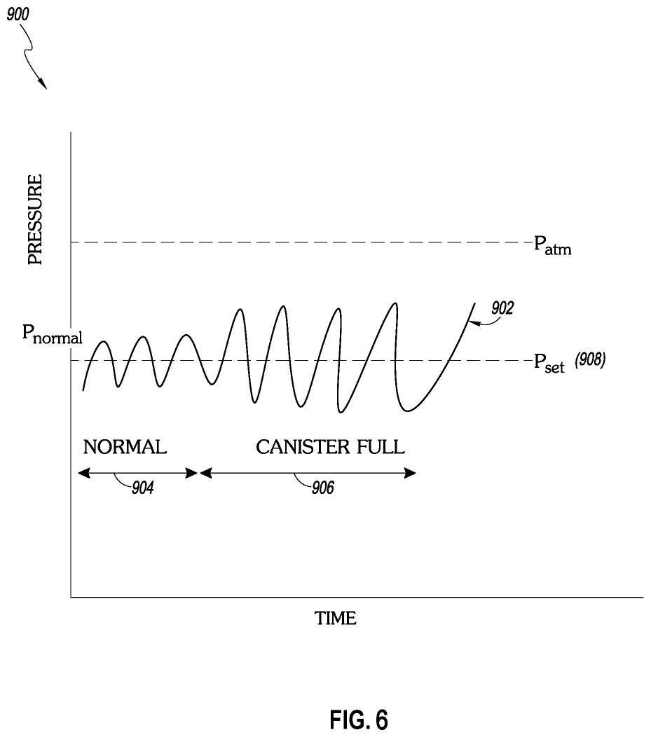

For example, the verification of detection by the main controller 310 can be related to canister full detection and notification (such as, canister full alarm). The pump controller 370 can perform canister full detection on short time intervals (such as, 1 second or less, 2 seconds, 3 second, 4 second, or 5 seconds or more) and provide notification to the main controller 310. The pump controller can determine whether the pressure in the fluid flow path satisfies a pressure threshold indicative of the canister being full. With reference to FIG. 6, pressure signals, which can be detected by one or more pressure sensors, are illustrated by the pressure curve 902. As is illustrated in a region 904, pressure in the fluid flow path varies or oscillates around a particular pressure set point 908 during normal operation. Region 906 illustrates pressure pulses in the flow path in presence of a blockage distal to the pump (such as, canister being full). As is illustrated in FIG. 6, such blockage cause an increase in the amplitude of the pressure signals. The pressure threshold indicative of the canister being full can be set to a value that would distinguish the change in the amplitude of the pressure signals between the regions 904 and 906. The pressure threshold indicative of the canister being full can be selected or determined based on one or more of the negative pressure set point and a current level of activity of the pump (which can be determined using the pump speed or the level of activity). The pump controller 370 can compare the amplitude of pressure signals (as measured by one or more pressure sensors) over one or more time intervals to a suitable pressure threshold indicative of the canister being full and, responsive to determining that the amplitude of the pressure signals satisfies (such as, exceeds) the threshold, provide a notification of canister full condition to the main controller 310.

The pump controller 370 can perform canister full detection by collecting a plurality of pressure sensor readings, each performed over a time duration (such as, 2 seconds). A number of readings of the plurality of readings, such as 25 sample periods out of 30 or any other suitable number, can be compared to the suitable pressure threshold indicative of the canister being full to determine if each indicates that the canister is full. This can performed by determining maximum and minimum pressure values captured over the time duration of a particular sample period. The values can be voltage values, current values, or any other suitable values that correspond to pressure. A difference between maximum and minimum values for a particular sample period corresponds to peak-to-through pressure (which is indicative of change in pressure pulse amplitude). If it is determined that the peak-to-through (or peak-to-peak) pressure for a particular sample period satisfies (such as, exceeds) the threshold, the pump controller 370 can determine that that the particular sample period indicates that the canister is full.

Canister full detection can be performed on a sliding window basis. For example, a sliding window of 25 out of 30 sample periods can be analyzed and if 25 sample periods are determined to indicate that the canister is full, the pump controller 370 can determine that the canister is full and provide a notification to the main controller 310. Assuming that the sample period is 2 seconds, using a sliding window of 25 out of 30 sample periods effectively results in determining whether change in pressure pulse amplitude satisfies the threshold for 60 seconds.

Additional details of canister full detection are disclosed in U.S. Pat. No. 10,744,239, which is incorporated by reference in its entirety.

The main controller 310 can perform verification or filtering of the canister full detection performed by the pump controller 370. For instance, the main controller 310 can verify that a particular number of canister full notifications has been received from the pump controller 370 before providing a canister full notification (or a canister full alarm) to the user. As an example, the main controller can verify that 5 or less, 10, 15, 20, 25, 30 or more consecutive canister full notifications have been received from the pump controller 370. For instance, assuming that the pump controller 370 provides a canister full notification in response to processing 60 seconds worth of pressure samples, five consecutive notifications may span 5 minutes, 10 consecutive notifications may span 10 minutes, and so on. In some cases, sliding window approach can be alternatively or additionally utilized, such as verifying that X canister full notifications over a duration of Y has been received from the pump controller 370. In response a successful verification, the main controller 310 can cause generation of the canister full alarm on the user interface. As a result, the main controller 310 can effectively perform filtering of the canister full detection by the pump controller 370 by delaying the assertion of the canister full alarm and thereby reducing the incidence of false alarms, improving patient safety, and increasing compliance, among others.

In some implementations, the verification or filtering for the canister full alarm can be performed when continuous negative pressure wound therapy is being provided. When intermittent negative pressure is being provided, the verification or filtering for the canister full alarm may not be performed in view of the changes in the negative pressure set point and the accompanying likelihood of intermittent assertion of the canister full alarm.

After the initial assertion of the canister full alarm, the main controller 310 can continue to provide notification of the alarm in response to continuing to receive canister full notifications from the pump controller 370. The canister full alarm can be cleared in response to not receiving a canister full notification from the pump controller 370 subsequent to the initial assertion of the canister full alarm.

While the canister full condition has been described, the disclosed approaches are applicable to other conditions, such as blockage, leakage, dressing full, overpressure, etc.

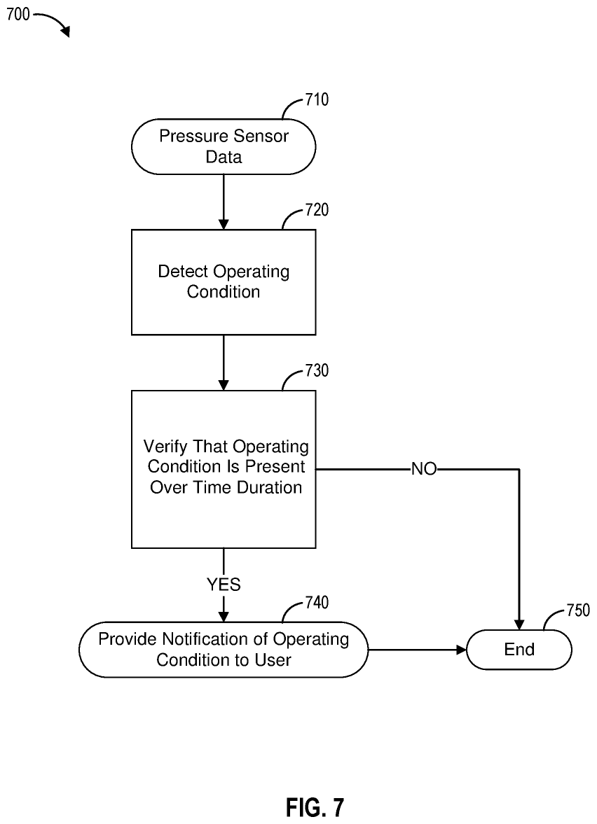

FIG. 7 illustrates a verification or filtering process 700 of an operating condition. The process 700 can be implemented by any of the negative pressure wound therapy systems disclosed herein. For instance, the process 700 can be implemented by the main controller 310 and the pump controller 370. In block 710, the process can receive data associated with provision of negative pressure wound therapy, such as pressure data from one or more pressure sensors. In block 720, the process 700 can detect presence of the operating condition and provide notification of the operating condition. For instance, blocks 710 and 720 can be performed by the pump controller 370, and the notification can be provided to the main controller 310. In block 730, the process 700 can perform filtering of the detection of the operating condition. As described herein, the process 700 can verify that the notification of the operating condition is provided over a time duration. In the verification is successful, the process 700 can transition to block 740 where notification of the operating condition (or alert) is provided to the user. Blocks 730 and 740 can be performed by the main controller 310.

If the verification is not successful, the process 700 can terminate in block 750. In some cases, in block 750, the main controller 310 can inform the pump controller 370 that detection of the operating condition was performed erroneously. This can cause the pump controller 370 to take one or more actions, including clearing the detection, resetting one or more parameters of the detection process, or the like. For instance, the main controller 310 can reset the pump controller 370 (such as, reset its memory).

In some implementations, the main controller 310 may be malfunctioning, and the pump controller 370 can take one or more remedial actions. The pump controller 370 can verify that it has correctly detected the operating condition despite the main controller 310 indicating that such detection was incorrect. For instance, the pump controller 370 can verify that it has correctly detected that the canister is full based on determining that the canister is full repeatedly over a time duration. On the other hand, the main controller 310 may be operating incorrectly in indicating to the pump controller 370 that the canister is not full. In such a situation, the pump controller 370 can attempt to reset the pump controller 310 (such as, reset its memory). The pump controller 370 can switch to a therapy mode that would keep the patient safe, for instance, by setting the negative pressure set point to a lower setting (such as, −25 mmHg) as any commands for adjusting therapy settings from the main controller 310 may be erroneous. The pump controller 370 can ignore or override certain commands from the main controller 310 that compromise patient safety, such as, a command to increase the negative pressure set point to a higher setting (such as, exceeding −75 mmHg). If possible, the pump controller 370 can provide an indication that the main controller 310 is malfunctioning.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound monitoring and/or treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. As another example, systems, devices, and/or methods disclosed herein can be used with a wound debridement system, patient monitoring system, or the like. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A negative pressure wound therapy system comprising:
a negative pressure source configured to provide, via a fluid flow path, negative pressure to a wound covered by a wound dressing;
a pressure sensor configured to measure a pressure in the fluid flow path;
a user interface configured to receive input from a user and provide a plurality of notifications to the user;
a first processor configured to operate the negative pressure source based on the pressure in the fluid flow path and detect an operating condition associated with provision of negative pressure; and
a second processor configured to operate the user interface and communicate with the first processor, the second processor being configured to:
based on the pressure in the fluid flow path, verify detection of the operating condition by the first processor;
provide, via the user interface, a notification of the operating condition to the user in response to verifying detection of the operating condition; and
override detection of the operating condition by the first processor in response to not verifying detection of the operating condition.

2. The system of claim 1, wherein providing the notification of the operating condition to the user in response to verifying detection of the operating condition improves sensitivity of the notification of the operating condition.

3. The system of claim 1, wherein overriding detection of the operating condition comprises not providing the notification of the operating condition to the user.

4. The system of claim 1, wherein the operating condition comprises at least one of:
a leak in the fluid flow path detected in response to the pressure in the fluid flow path not satisfying a pressure threshold over a duration of time;
a blockage in the fluid flow path detected in response to flow in the fluid flow path no longer satisfying a flow threshold; or
a canister full condition indicative of a level of fluid in a canister satisfying a fluid level threshold.

5. The system of claim 4, wherein the first processor is configured to detect the canister full condition in response to a determination that the pressure in the fluid flow path over a duration of time satisfies a pressure threshold.

6. The system of claim 1, further comprising a housing supporting the negative pressure source and the first processor, wherein the second processor is supported by the housing or is remote to the housing.

7. The system of claim 1, wherein the operating condition is communicated to the second processor via a serial interface.

8. The system of claim 1, wherein the operating condition is detected periodically within a specified duration of time.

9. The system of claim 1, wherein the second processor is configured to verify detection of the operating condition further based on a detection of one or more environmental changes.

10. The system of claim 9, wherein the one or more environmental changes comprises a change in altitude.

11. The system of claim 1, wherein in response to not verifying detection of the operating condition, the second processor is further configured to cause the first processor to clear detection of the operating condition.

12. The system of claim 1, wherein in response to not verifying detection of the operating condition, the second processor is further configured to reset the first processor.

13. A kit comprising the system of claim 1 and at least one of the wound dressing or a canister configured to be positioned in the fluid flow path and further configured to store fluid aspirated from the wound.

14. (canceled)

15. A negative pressure wound therapy system comprising:
a negative pressure source configured to provide, via a fluid flow path, negative pressure to a wound covered by a wound dressing;
a pressure sensor configured to measure a pressure in the fluid flow path;
a user interface configured to receive input from a user and provide a plurality of notifications to the user;
a first processor configured to operate the negative pressure source based on the pressure in the fluid flow path and detect an operating condition associated with provision of negative pressure; and
a second processor configured to verify detection of the operating condition by the first processor and provide a notification of the operating condition by:
upon expiration of a first time duration, receiving from the first processor an indication that the operating condition has been detected during the first time duration; and
via the user interface, providing the notification of the operating condition to the user in response to verifying that a plurality of indications have been received from the first processor during a second time duration that spans a plurality of first time durations.

16. The system of claim 15, wherein providing the notification of the operating condition to the user in response to verifying that the plurality of indications have been received from the first processor during the second time duration reduces incidence of false alarms.

17. The system of claim 15, further comprising a canister configured to be positioned in the fluid flow path and further configured to store fluid aspirated from the wound, wherein the operating condition comprises detection that the canister is full.

18. The system of claim 17, wherein the first processor is configured to detect that the canister is full in response to the pressure over the first time duration satisfying a pressure threshold.

19. The system of claim 17, wherein the second processor is configured to continue provision of the notification of the operating condition in response to receiving from the first processor the indication subsequent to expiration of the second time duration.

20. The system of claim 17, wherein the second processor is configured to stop provision of the notification of the operating condition in response to no longer receiving from the first processor the indication subsequent to expiration of the second time duration.

21-24. (canceled)

25. The system of claim 1, wherein the second processor is configured to:
upon expiration of a first time duration, receive from the first processor an indication that the operating condition has been detected during the first time duration; and
via a user interface, provide the notification of the operating condition to the user in response to verifying that a plurality of indications have been received from the first processor during a second time duration that spans a plurality of first time durations.
